(19) United States
(12) Patent Application Publication
(10) Pub. No.: US 2010/0055215 A1
(43) Pub. Date: Mar. 4, 2010

(54) PROCESS TO LOSE WEIGHT, DRAIN UNWANTED BODY FATS AND OTHER TOXINS

(76) Inventor: Juanito B. Calagui, Passaie, NJ (US)

Correspondence Address: Mr. Walter J. Tencza Jr., Suite 210, 100 Menlo Park, Edison, NJ, 08837, US

(21) Appl. No.: 12/615,554

(22) Filed: Nov. 10, 2009

Related U.S. Application Data
(63) Continuation-in-part of application No. 12/413,881, filed on Mar. 30, 2009.
Application No. 12/413,881 is a continuation-in-part of application No. 12/107,137, filed on Apr. 22, 2008.
Application No. 12/107,137 is a continuation-in-part of application No. 12/028,947, filed on Feb. 11, 2008.
Application No. 12/028,947 is a continuation-in-part of application No. 11/927,936, filed on Oct. 30, 2007.
Application No. 11/927,936 is a continuation-in-part of application No. 11/306,610, filed on Jan. 4, 2006, now abandoned.
Application No. 11/306,610 is a continuation-in-part of application No. 10/601,684, filed on Jun. 23, 2003, now abandoned.

(51) Int. Cl. A61K 36/82; A61P 3/04

(52) U.S. Cl. 424/729

(57) ABSTRACT

A method is disclosed comprising placing about thirty-two fluid ounces of water into a receptacle, placing the receptacle with water on a burner and bringing the water to a boil, ceasing to heat the water, adding about one tablespoonful or five grams of tea beverage flavorant to the water; and brewing the combination of the water and the tea beverage flavorant for about three to five minutes. The method may further include filtering out any solid tea in the combination of water and tea beverage flavorant, and consuming the combination of the water and the tea beverage flavorant.

### CROSS REFERENCE TO RELATED APPLICATIONS

The present application is a continuation in part of and claims the priority of U.S. patent application Ser. No. 12/413,881, entitled “PROCESS TO LOSE WEIGHT, DRAIN UNWANTED BODY FATS AND OTHER TOXINS”, filed on Mar. 30, 2009, inventor and applicant Juanito B. Calagui, which is a continuation in part of and claims the priority of U.S. patent application Ser. No. 12/107,137, entitled “PROCESS TO LOSE WEIGHT, DRAIN UNWANTED BODY FATS AND OTHER TOXINS”, filed on Apr. 22, 2008, inventor and applicant Juanito B. Calagui, which is a continuation in part of and claims the priority of U.S. patent application Ser. No. 12/028,947, entitled “PROCESS TO LOSE WEIGHT, DRAIN UNWANTED BODY FATS AND OTHER TOXINS”, filed on Feb. 11, 2008, inventor and applicant Juanito B. Calagui, which is a continuation in part of and claims the priority of U.S. patent application Ser. No. 11/927,936, entitled “PROCESS TO LOSE WEIGHT, DRAIN UNWANTED BODY FATS AND OTHER TOXINS”, inventor and applicant Juanito B. Calagui, filed on Oct. 30, 2007, which is a continuation in part of and claims the priority of patent application Ser. No. 11/306,610, entitled “PROCESS TO LOSE WEIGHT, DRAIN UNWANTED BODY FATS AND OTHER TOXINS”, inventor and applicant Juanito B. Calagui, filed on Jan. 4, 2006, which is a continuation in part of and claims the priority of patent application Ser. No. 10/601,684 entitled “PROCESS TO LOSE WEIGHT, DRAIN UNWANTED BODY FATS AND OTHER TOXINS”, inventor and applicant Juanito B. Calagui, filed on Jun. 23, 2003, and patent application Ser. No. 10/662,608 entitled “PROCESS TO LOSE WEIGHT, DRAIN UNWANTED BODY FATS AND OTHER TOXINS”, inventor and applicant Juanito B. Calagui, filed on Sep. 15, 2003. The present application claims the priority of all of the above patent applications including Ser. Nos. 12/413,881; 12/107,137; 12/028,947; 11/927,936; 11/306,610; 10/601,684; and 10/662,608.

### FIELD OF THE INVENTION

This invention relates to improved methods and apparatus concerning losing weight.

### BACKGROUND OF THE INVENTION

It is very common practice for kids, teenagers and adults to keep eating and eating whenever foods are available. This causes them to become overweight. Overweight individuals use diet drinks, diet pills or join physical fitness centers to lose weight and to drain unwanted body fats. However, many of these options do not result in loss of weight or fat for many overweight people.

The human body is a living structure of multiple mechanical assembly links which are associated to each other. The above-mentioned linkage and association of the assembly within the human body are through the blood vessels and within these channels malfunctions and irregularities are mostly caused by the buildup of body fats. The buildup of body fats causes a wide range of damage to the organs.

Generally speaking, medical research indicates that fat is the cause of many health care problems.

### SUMMARY OF THE INVENTION

The present invention in one or more embodiments provides a process which cleanses and dissolves foreign bodies within the human body.

A method is disclosed comprising the steps of placing about thirty-two fluid ounces of water into a receptacle, placing the receptacle with water on a burner and bringing the water to a boil, ceasing to heat the water, adding about one tablespoonful or five grams of tea beverage flavorant to the water; and brewing the combination of the water and the tea beverage flavorant for about three to five minutes. The method may further include filtering out any solid tea in the combination of water and tea beverage flavorant, and consuming the combination of the water and the tea beverage flavorant.

The temperature of the combination of the water and the tea beverage flavorant at the time that the water and the tea beverage flavorant are consumed may be between one hundred forty and one hundred sixty degrees Fahrenheit. The combination of the water and the tea beverage flavorant may be placed into a disposable plastic bottle. The combination of the water and the tea beverage flavorant may be called the mixture.

The steps of the method, including the consumption of the mixture, may be performed, twice a day, typically once after breakfast and once after 12:00 noon. The inventor has found that such a regimen results in a good overall cleansing effect and in a loss of weight.

A mixture of water and a tea beverage flavorant together in the proportions of about thirty-two fluid ounces for about every one tablespoonful or every five grams of tea beverage flavorant can be placed into a disposable plastic bottle. The mixture can be later heated and consumed.

The present invention also provides an apparatus comprising a disposable plastic bottle, which has a mixture of water and a tea beverage flavorant in the proportions of about thirty-two fluid ounces of water for about every one tablespoonful or every five grams of tea beverage flavorant, located inside the disposable plastic bottle.

The indication of no body fat appears by looking at the result of an individual's bowel movement in a toilet bowl (i.e. by looking at the feces and/or urine resulting from the bowel movement).

A method in accordance with an embodiment of the present invention dissolves and drains the buildup of body fats in the organs of a person, which helps a person to enjoy a better life.

In one or more embodiments of the present invention a bowel movement in response to drinking or ingesting a mixture in accordance with an embodiment of the present invention, causes draining of fats from a person's body. In accordance with a method of an embodiment of the present invention a person should observe and/or test by medical tests feces and/or urine from their bowel movements to see if the feces and/or urine contain fats.

If a user of a mixture in accordance with an embodiment of the present invention, is approximately twenty to thirty-nine years old, the user should drink or ingest the mixture every day, twice a day, for a first period of time, until there is no presence of fat in a bowel movement of the individual, which may be determined by observation or medical testing of feces and/or urine. After no presence of fat is detected, how, the person should continue drinking or ingesting the mixture every day, twice a day for a second period of time. The second period of time may be about six days for a person twenty to thirty-nine years old and about twelve days for a person forty-years old or older. After the second period of time of detecting no body fat or substantially no body fat in all bowel movements then the person should completely stop taking the mixture to avoid dehydration. In one embodiment, the person can start taking the mixture again after two headaches have occurred every day for at least four consecutive days. In one embodiment, the person should wait at least several years before taking the mixture again after stopping.

If a user of a mixture in accordance with an embodiment of the present invention is approximately forty years old or older, the user should drink or ingest the mixture every day, twice a day, for a first period of time, until there is no presence of fat in feces or urine resulting from a bowel movement of the individual. After no presence of fat is detected, the person should continue drinking or ingesting the mixture every day, twice a day for a second period of time, typically about six days for a twenty to thirty-nine year old person and about twelve days for a person forty years old or older. After twelve days of detecting no body fat or substantially no body fat in all feces or urine resulting from bowel movements then the person should completely stop taking the mixture to avoid dehydration.

In one embodiment, draining unwanted body fats and other toxins may follow thirty-three to thirty-six days after the start of the process of drinking or ingesting the mixture. The dissolving and draining of fats may continue for eighteen months or more and the user should observe his or her bowel movements, and determine by the bowel movements when no presence of fat appears, which can be determined by observation or by medical testing of feces and/or urine of the individual. When no fat appears in feces or urine of bowel movements in about six days for a twenty to thirty-nine year old or about twelve days for a forty year old then the user should stop the process of ingesting the mixture in order to prevent dehydration and prevent other possible negative health effects such as cancer.

After the six or twelve consecutive days of detecting no fat or substantially no fat in feces and urine of the individual, the individual should completely stop taking the mixture. The individual can resume taking the mixture when after the individual has two to three headaches for a period of at least four consecutive days.

In accordance with an embodiment of the present invention “Tikuanjin Tea” typically made in Taiwan which is a trademarked type of tea is especially useful and may have the optimum capacity to dissolve and drain the buildup of body fats and to prevent terminal diseases and cancer.

### BRIEF DESCRIPTION OF THE DRAWINGS

FIG. 1 shows a flow chart of a method in accordance with an embodiment of the present invention;

FIG. 2 shows a disposable plastic bottle which includes a mixture of water and tea beverage flavorant;

FIG. 3 shows a flow chart of a method in accordance with another embodiment of the present invention;

FIG. 4 shows a flow chart of a method in accordance with another embodiment of the present invention; and

FIG. 5 shows a diagram of a time line in accordance with the method of FIG. 4.

### DETAILED DESCRIPTION OF THE DRAWINGS

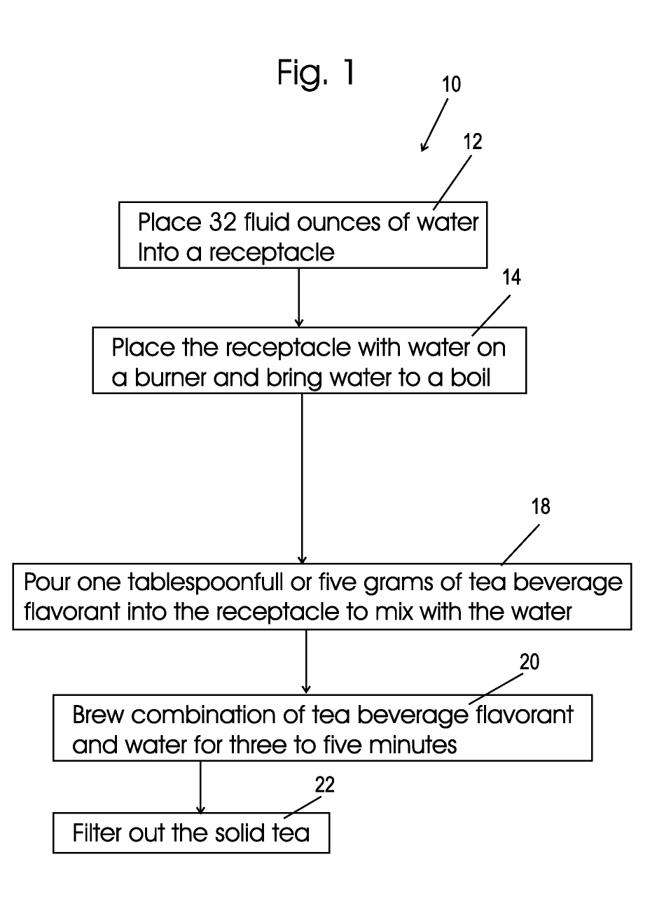

FIG. 1 shows a flow chart 10 of a method in accordance with an embodiment of the present invention. The method begins with placing thirty-two fluid ounces of water into a receptacle at step 12. The receptacle, with the water, is then placed on a burner and brought to a boil at step 14. At step 18 one tablespoonful of tea beverage flavorant or five grams is poured into the receptacle to mix with the water. At step 20 the combination of water and tea beverage flavorant is brewed for three to five minutes. At step 22 the solid tea in the combination of water and tea beverage flavorant is filtered out. The remaining combination or drink can be consumed by an individual.

The remaining drink should be at 140-160 degrees Fahrenheit when consumed by an individual for the best cleansing effect for dissolving foreign bodies and for loss of weight generally. The applicant has tested a remaining drink with the temperature between 140-160 degrees Fahrenheit versus drinks of lower temperatures and has found that the temperature range of 140-160 degrees Fahrenheit is optimum for dissolving toxins and cleansing of the body and for loss of weight.

A method in accordance with an embodiment of the present invention may include repeating the procedure shown in FIG. 1, everyday, twice a day, typically once after breakfast and once before dinner.

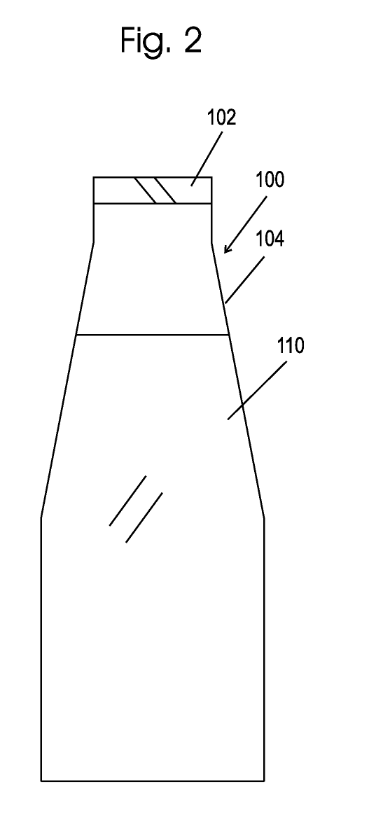

In addition, the method may additionally include placing the mixture of a ratio of 32 fluid ounces of water to one tablespoonful or five grams of tea flavorant into a disposable plastic bottle, which can be sold to a purchaser. FIG. 2 shows a disposable plastic bottle 100. The bottle 100 is transparent in this example and includes a cap 102 and a body portion 104 or receptacle for holding liquid. The body portion 104 in FIG. 2 holds a mixture of water and tea beverage flavorant 110 in the proportions of thirty-two fluid ounces of water for every one tablespoonful or every five grams of tea beverage flavorant. The cap 102 provides a seal so that the mixture 110 is held in a sealed, enclosed chamber inside the bottle 100. The mixture 110 in the disposable plastic bottle 100 can be readily heated to the appropriate temperature and consumed.

The disposable plastic bottle may have a capacity of thirty-two fluid ounces.

In one embodiment, draining unwanted body fat and other toxins may follow thirty-three to thirty-six days after the process has been started.

The present invention in one or more embodiments, introduces a new process to lose weight, drain unwanted body fats and other toxins to cleanse within a living structure the human body to be healthy, pain free and cancer free.

The human body is a living structure of multiple mechanical assembly links and associated to the function of each or the other assembly. The above-mentioned linkage and association of the assembly within the human body are through the blood vessels or flowing channels and within these channels malfunctions and other irregularities are mostly caused by the buildup of fats likewise, the buildup of fats will eventually develop into the blockade of the blood vessels or flowing channels and the blockade will cause the tissue fluid to lose contact within an organ to dehydrate and collapse or die and in the end decaying tissue fluid will then turn into toxin to cause cancer. The new process is to dissolve and prevent the buildup of fats within tissue fluid and the blood vessels or flowing channels of a human being and also to dissolve pockets of fats in other portions of the body of a human being. The process in accordance with one or more embodiments of the present invention act as a cleansing agent and is effective to drain unwanted body fats and other toxins to help an individual to be healthy and to enjoy a better life.

The present invention in one or more embodiments has a beneficial effect on the heart, the respiratory system, and helps individuals to be cancer free with the use of a new process in accordance with one or more embodiments of the present invention. The malfunctions and irregularities of the blood vessels are most often caused by the buildup of fats that slow down the deliveries of nutrient-rich blood and dehydrate the muscles and linkages to the organs.

One or more embodiments of the present invention can be used for medical and dental reasons and as preventive maintenance to help a human being to be pain free, cancer free, and healthy. It is also beneficial for people suffering from asthma. It is often difficult to detect why sicknesses, such as cancer and diabetes occur, and embodiments of the present invention are useful in ameliorating those sicknesses.

The mixture of tea is typically heated to a temperature of between one hundred forty and one hundred sixty degrees Fahrenheit.

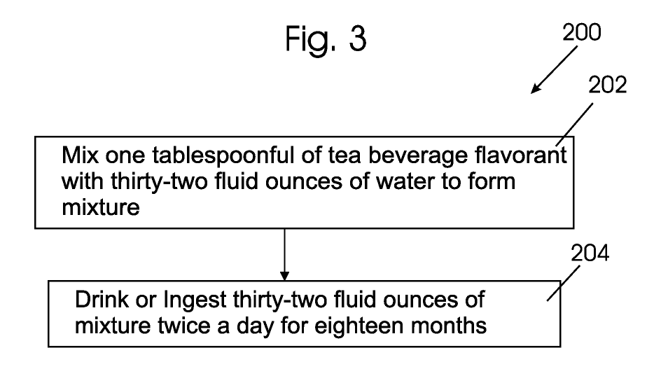

FIG. 3 shows a flow chart 200 of a method in accordance with another embodiment of the present invention. At step 202 one tablespoonful of tea beverage flavorant is mixed with about thirty-two fluid ounces of water to form a mixture and the temperature of the mixture should be between one hundred forty and one hundred sixty degrees Fahrenheit. At step 204 a human being would then drink or ingest about thirty-two fluid ounces of the mixture twice a day for eighteen months and stopped to prevent dehydration.

One or more embodiments of the present invention help prevent and stop cancer and the Tikuanyin (trademarked) tea has a cleansing agent to cleanse the blood vessels and the organs that were partly coated on the inside with fat. The build up of fat causes a wide range of damage to the organs and the depositing of fat into the organs continues during a person's entire life. A bowel movement demonstrates the draining of fat from the human body. A loose bowel movement drains first without the presence of fat. The start of the draining of fat from the human body will follow thirty-three to thirty-six days after the initial regimen of drinking or ingesting a mixture in accordance with an embodiment of the present invention into the human body, such as the mixture of one tablespoonful of tea beverage flavorant mixed with about thirty-two fluid ounces of water, at a temperature of about one hundred forty to one hundred sixty degrees Fahrenheit.

If the user of a mixture in accordance with an embodiment of the present invention, is twenty to thirty-nine years old, the user of the mixture needs to drink or ingest the mixture for twenty months and then should stop to prevent dehydration. Individuals forty years old and older should drink the mixture for eighteen months and stopped to prevent dehydration.

The new process is the only effective procedure to drain unwanted body fats and other toxins to help an individual prevent the mechanics that cause osteoporosis, AIDS (Acquired Immune Deficiency Syndrome), epilepsy, lupus erythematosus, leukemia, gall bladder cancer, breast cancer, malaria, autism, colon cancer, and pancreatic cancer and keeping the channels leading to the reproductive organs clean, to keep the reproductive organs always ready.

The depositing of fat into the organs continues during a person's entire life. This is at least in part because, fat is always a portion of the blood pumped from the heart into the organs to feed the organs. The fat causes a wide range of damage to the organs.

An individual user of a mixer in accordance with one or more embodiments of the present invention, such as the mixture 110, may ingest or drink two bottles worth (i.e. sixty-four fluid ounces), each bottle identical or similar to bottle 100, of the mixture 110 twice a day. The mixture, such as mixture 110, may be taken once in the morning and once after 12:00 noon. The temperature of the mixture, such as mixture 110, at the time of ingestion or drinking in a preferred embodiment of the present invention should be between one hundred and forty and one hundred and sixty degrees Fahrenheit. A thermometer can be used to check the temperature of the mixture, such as mixture 110, which can be called “magic tea”. A mixture in accordance with one or more embodiments of the present invention, particularly at a temperature in the range of approximately one hundred forty to one hundred sixty degrees, is used to dissolve and drain the buildup of fats. The mixture, such as 110, is taken, twice a day as referred to above, every day, and a person watches his or her bowel movements to determine when there is no fat in a bowel movement. When no fat is detected in any bowel movement for six consecutive days for an individual in the range of twenty to thirty-nine years old, in accordance with an embodiment of the present invention, that individual would stop ingesting the mixture, to prevent dehydration. When no fat is detected in any bowel movement for twelve consecutive days for an individual forty years old and older, in accordance with an embodiment of the present invention, that individual would stop ingesting the mixture, to prevent dehydration.

The above mentioned procedures in one or more embodiments of the present invention, to follow a preventive measure and the process pay as you go is adoptable to everybody documented or undocumented. An individual can use a mixture in accordance with one or more embodiments of the present invention, such as mixture 110, as an organ cleaner to keep a diabetic count low and no health insurance is necessary. The individual can keep his or her organs clean by using the mixture 110, in accordance with one or more methods of embodiments of the present invention previously described, to prevent malfunction, and irregularities of the organs, and in the end will prevent cancer.

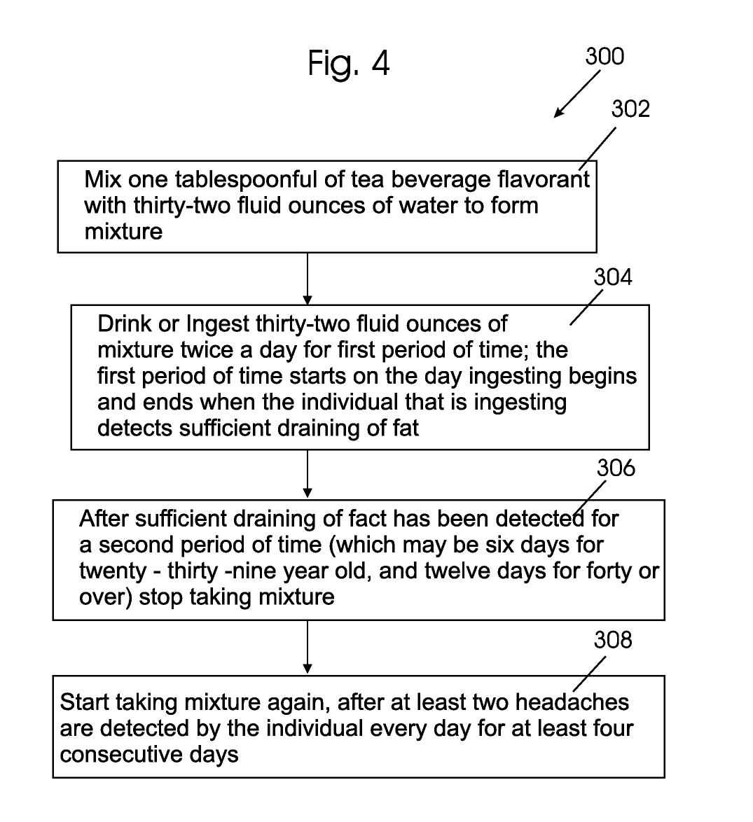

FIG. 4 shows a flow chart 300 of another method in accordance with an embodiment of the present invention. At step 302, an individual would mix one tablespoonful of tea beverage flavorant with thirty-two fluid ounces of water to form a mixture. The tea beverage flavorant may be Tikuanjin tea (trademarked) in accordance with one embodiment of the present invention. At step 304, the individual would drink or ingest thirty-two fluid ounces of mixture twice a day for a first period of time. The first period of time starts on the day when ingesting or drinking begins and ends when the individual that is ingesting detects sufficient draining of fat. The sufficient draining of fat, may be a detection no fat or very little fat in feces and/or urine of a bowel movement of the individual which has been excreted by the individual into a toilet bowl.

At step 306, after a sufficient draining of fat has been detected for a second period of time (which may be six days for a twenty to thirty-nine year old, and twelve days for a person forty years old or older) then the person should stop ingesting, drinking or in any way taking the mixture. The person can start taking the mixture again when the person has at least two headaches at step 308, for at least four consecutive days.

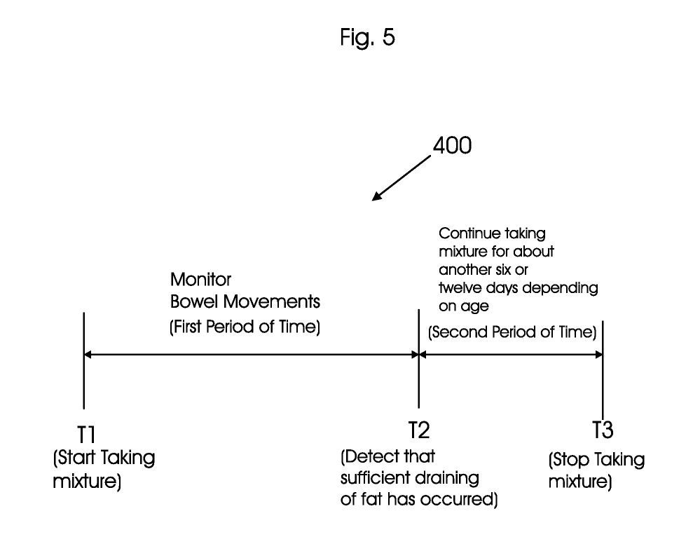

FIG. 5 shows a diagram 400 of a time line in accordance with the method of FIG. 4. At time T1 a human being starts to ingest or take a mixture as previously described in one or more embodiment of the present invention. The human being or individual may take the mixture two times a day for a first period of time as referred to in FIGS. 4 and 5. During that first period of time, the individual may monitor his or her bowel movements, such as by examining his or her own feces and/or urine in a toilet bowl after his or her own bowel movement, to determine if fat has been sufficiently drained from the person. The feces and/or urine of the individual may be oily when there is fat present, and when there is little fat or no fat present, the feces and/or urine may not be oily. Alternatively, a medical test may be performed by a medical professional on the feces and/or urine of the person to determine if sufficient fat has been drained from the individual.

In at least one embodiment sufficient draining of fat may be the detection of virtually no fat or not fact in the feces and/or urine of the person, at a time T2 as shown in FIG. 5. After the person has detected a sufficient draining of fat, the person, in at least one embodiment, should continue ingesting and/or drinking the mixture for a second time period of about six days for a twenty to thirty-nine year old or about twelve days for a forty year old or over. The first period of time shown in FIG. 5 and the second period of time shown in FIG. 5 run continuously and consecutively. At time T3 (which is about six or twelve days after time T2), the individual should completely stop taking the mixture to avoid dehydration and other possible negative health consequences. In at least one embodiment, the person should completely stop taking the mixture for at least one year, and preferably several years, such as ten to fifteen years or more. Because the fat has been drained from the individual, it should take many years for the buildup of fat to occur again, if at all. In at least one embodiment, the individual can restart taking the mixture if necessary at a later time, such as after time T3, and after the individual has at least two headaches every day for a period of at least four consecutive days.

Although the invention has been described by reference to particular illustrative embodiments thereof, many changes and modifications of the invention may become apparent to those skilled in the art without departing from the spirit and scope of the invention. It is therefore intended to include within this patent all such changes and modifications as may reasonably and properly be included within the scope of the present invention's contribution to the art.

I claim:

1. A method comprising the steps of:
placing about thirty-two fluid ounces of water into a receptacle;
placing the receptacle with water on a burner and bringing the water to a boil;
ceasing to heat the water;
adding about one tablespoonful of tea beverage flavorant to the water; and
brewing the combination of the water and the tea beverage flavorant for about three to five minutes; and
consuming the combination of the water and the tea beverage flavorant;
repeating the above steps every day for a first period of time until it is determined that sufficient fat has been drained from an individual who has executed the step of consuming the combination;
if it is determined that sufficient fat has been drained from the individual, continuing to consume the combination every day for a second period of time;
after the second period of time, ceasing to consume the combination for at least one year.

2. The method of claim 1 wherein
the determination of whether sufficient fat has been drained from the individual who has executed the step of consuming the combination is based on an observation of urine or feces from a bowel movement of the individual.

3. The method of claim 1 wherein
the second period of time is about six days for an individual that is approximately twenty to thirty nine years old.

4. The method of claim 1 wherein
the second period of time is about twelve days for an individual that is approximately forty years old or older.

5. The method of claim 1 further comprising
filtering out any solid tea in the combination of water and tea beverage flavorant after the step of brewing the combination and before the step of consuming the combination.

6. The method of claim 1 wherein
the temperature of the combination of the water and the tea beverage flavorant at the time that the water and the tea beverage flavorant are consumed is between one hundred forty and one hundred sixty degrees Fahrenheit.

7. The method of claim 1 further comprising
placing the combination of the water and the tea beverage flavorant into a disposable plastic bottle, after the step of brewing the combination and before the step of consuming the combination.

8. The method of claim 1 further comprising
performing the previous steps up to and including the step of consuming the combination once after breakfast.

9. The method of claim 1 further comprising
performing the previous steps up to and including the step of consuming the combination once before dinner.

10. The method of claim 1 further comprising
performing the previous steps up to and including the step of consuming the combination once after breakfast and once after noon time.

11. A method comprising the steps of
mixing water and a tea beverage flavorant together in the proportions of about thirty-two fluid ounces of water for about every one tablespoonful of tea beverage flavorant to form a mixture;
placing the mixture in a disposable plastic bottle; and
consuming the mixture;
repeating the above steps every day for a first time period, until an individual that has executed the step of consuming the mixture detects a sufficient draining of fat from his or her body;
if it is determined that sufficient fat has been drained from the individual's body, continuing to consume the combination every day for a second period of time;
after the second period of time, ceasing to consume the combination until the individual has at least two headaches for at least four consecutive days.

12. A method comprising the steps of
mixing water and a tea beverage flavorant together in the proportions of about thirty-two fluid ounces for about every one tablespoonful of tea beverage flavorant to form a mixture;
and consuming the mixture;
repeating the above steps every day for a first period of time until an individual that is executing the step of consuming the mixture detects a sufficient draining of fat from his or her body;
if it is determined that sufficient fat has been drained from the individual's body, continuing to consume the combination every day for a second period of time;
after the second period of time, ceasing to consume the combination until the individual has at least two headaches for at least four consecutive days.

13. A method comprising the steps of:
placing about thirty-two fluid ounces of water into a receptacle;
placing the receptacle with water on a burner and bringing the water to a boil;
ceasing to heat the water;
adding about five grams of tea beverage flavorant to the water; and
brewing the combination of the water and the tea beverage flavorant for about three to five minutes; and
consuming the combination; and
repeating the above steps every day for a first period of time until an individual that is executing the step of consuming the mixture detects a sufficient draining of fat from his or her body;
if it is determined that sufficient fat has been drained from the individual's body, continuing to consume the combination every day for a second period of time;
after the second period of time, ceasing to consume the combination for at least one year.

14. The method of claim 13 further comprising
filtering out any solid tea in the combination of water and tea beverage flavorant, after the step of brewing the combination and before the step of consuming the combination.

15. The method of claim 13 wherein
the temperature of the combination of the water and the tea beverage flavorant at the time that the water and the tea beverage flavorant are consumed is between one hundred forty and one hundred sixty degrees Fahrenheit.

16. The method of claim 13 further comprising
placing the combination of the water and the tea beverage flavorant into a disposable plastic bottle after the step of brewing the combination and before the step of consuming the combination.

17. A method comprising the steps of
mixing water and a tea beverage flavorant together in the proportions of about thirty-two fluid ounces for about every five grams of tea beverage flavorant to form a mixture;
consuming the mixture;
repeating the above steps every day for a first time period until an individual that is executing the step of consuming the mixture detects a sufficient draining of fat from his or her body;
if it is determined that sufficient fat has been drained from the individual's body, continuing to consume the mixture every day for a second period of time;
after the second period of time, ceasing to consume the combination for at least one year.

18. The method of claim 17 further comprising
placing the mixture in a plastic disposable bottle prior to consuming.

19. A method comprising
drinking and ingesting a mixture into a human body at least twice a day, including once after breakfast and once after noon time, every day for a first time period;
wherein the mixture is comprised of water and tea beverage flavorant in the ratio of about one tablespoonful of tea beverage flavorant to thirty-two fluid ounces of water; and further comprising
repeating the above steps every day for a first time period until an individual that is executing the step of drinking and ingesting the mixture detects a sufficient draining of fat from the human body;
if it is determined that sufficient fat has been drained from the human body of the individual, continuing to consume the mixture every day for a second period of time;
after the second period of time, ceasing to consume the combination for at least one year;
wherein the mixture has a temperature of approximately between one hundred forty and one hundred sixty degrees Fahrenheit.
